(19) United States
(12) Patent Application Publication
(10) Pub. No.: US 2019/0388329 A1
(43) Pub. Date: Dec. 26, 2019

(54) COSMETIC COMPOSITION HAVING MODELING PROPERTIES FOR A TEMPORARY RESHAPING OF KERATIN-CONTAINING FIBERS

(71) Applicant: Henkel AG & Co. KGaA, Duesseldorf (DE)

(72) Inventors: NORA KOOPMANN, Hamburg (DE); MARCUS NOLL, Quickborn (DE); SANDRA BRANDT, Pinneberg (DE)

(21) Appl. No.: 16/451,770

(22) Filed: Jun. 25, 2019

(30) Foreign Application Priority Data
Jun. 26, 2018 (DE) 10 2018 115 298.1

(51) Int. Cl. A61K 8/81; A61K 8/92; A61K 8/36; A61K 8/34; A61K 8/31; A61Q 5/06

(52) U.S. Cl. CPC A61K 8/8135; A61K 8/8176; A61K 8/922; A61Q 5/06; A61K 8/342; A61K 8/31; A61K 8/361

(57) ABSTRACT

The present disclosure relates to a cosmetic composition for the temporary reshaping of keratinic fibers, in particular human hair, containing, based on its total weight, a film former, a wax, a cosmetic oil, a branched or unbranched hydrocarbon and an emulsifier. The present disclosure also relates to a method for the temporary reshaping of the hair using the composition as contemplated herein. Furthermore, the use of the cosmetic composition for temporary reshaping of the hair is also disclosed.

### CROSS-REFERENCE TO RELATED APPLICATION

This application claims priority to German Patent Application No. 10 2018 115 298.1, filed Jun. 26, 2018, which is incorporated herein by reference in its entirety.

### TECHNICAL FIELD

The present disclosure relates to cosmetic compositions for the temporary reshaping of keratin-containing fibers, comprising a film former, a wax, a cosmetic oil and a branched or unbranched hydrocarbon. Furthermore, the present disclosure relates to the use of the cosmetic compositions for a temporary reshaping and/or a shape fixing of rigid hair, in particular beard hair, and a method for the temporary reshaping of keratinic fibers using the cosmetic compositions.

### BACKGROUND

Cosmetic agents which are used for a permanent or temporary shaping of the hair play an important role in cosmetics. The temporary shaping, which is intended give a good hold without impairing the healthy appearance of the hair, such as its shine, can be achieved, for example, by hair sprays, hair waxes, hair gels, hair mousses, etc. The cosmetic agents should be compounded depending on these application forms.

An important feature of an agent for the temporary reshaping of keratinic fibers is to give the treated fibers the strongest possible hold in the generated shape. If the keratinic fibers are human hair, this is also referred to as a strong hairstyle hold or the high degree of hold of the cosmetic agent.

The properties expected of cosmetic agents are subject to constant change. Fashionable consumers expect changing properties from time to time. For example, the property of a cosmetic to produce volume or shine is sometimes less significant. The properties that an agent produces are also dependent on the hair type. In addition to hair color, human hair differs in its ability to be strained by hair treatments, to be smooth or curly in nature, and also particularly in the properties of thickness and rigidity of the hair. The thickness and the rigidity of the hair play a special role in the temporary reshaping, since the resistance to the desired shape of the hair is great.

Beard hair is especially rigid and resists a temporary shaping. Currently, full beards and beard hairstyles are modern and the need for cosmetic agents for beard care and shaping is great. However, the cosmetic treatment of beards is not only subject to special requirements due to the rigidity of the beard hair. The requirements in terms of feel and shine can differ from those of the main hair. Nevertheless, there is a desire to style or dress the beard and hair with the help of a single styling agent. Thus, a special desire of some customers is to use a single cosmetic agent for use on scalp hair and beard. Such “2 in 1” products are increasingly in demand and must meet very specific requirements.

Few cosmetic agents are known in the prior art which have been developed for a temporary shaping of rigid hair, in particular for beard hair. Thus, there is a need for cosmetic agents for the temporary shaping of rigid hair, in particular beard hair.

### BRIEF SUMMARY

According to an embodiment of the present disclosure, a cosmetic composition for the temporary reshaping of keratinic fibers comprises, based on the total weight of the cosmetic composition, at least one film former in a total amount of from about 1 to about 10% by weight, at least one wax in a total amount of from about 3 to about 15% by weight, at least one cosmetic oil in a total amount of from about 0.5 to about 12% by weight, at least one branched or unbranched hydrocarbon in a total amount of from about 5 to about 12% by weight, at least one emulsifier in a total amount of from about 1 to about 4% by weight, and water in a total amount of from about 30 to about 50% by weight.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object underlying the present disclosure is to provide a cosmetic composition which is suitable for the temporary reshaping of keratin-containing fibers, in particular rigid hairs, especially beard hairs, wherein the particularities of rigid hairs, in particular beard hair, are taken into account with regard to the styling properties. Furthermore, the object underlying the present disclosure is to provide a cosmetic composition which is suitable to care for and style scalp and beard hair alike with an agent.

A first subject of the present disclosure is a cosmetic composition for the temporary reshaping of keratinic fibers, in particular human hair, containing, based on its total weight, at least one film former in a total amount of from about 1 to about 10% by weight, preferably from about 4 to about 9% by weight, more preferably from about 6 to about 8% by weight, at least one wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10, more preferably from about 5 to about 8% by weight, at least one branched or unbranched hydrocarbon in a total amount of from about 5 to about 12% by weight, preferably from about 6 to about 10, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight.

The cosmetic composition as contemplated herein is suitable for styling and caring for beard hair. As the cosmetic composition is also suitable for styling and care of the scalp hair, the cosmetic composition is a 2 in 1 formulation, that is, it provides a composition which is suitable for two purposes.

According to the present disclosure, the cosmetic composition comprises a film former in a total amount of from about 1 to about 10% by weight, preferably from about 4 to about 9% by weight, more preferably from about 6 to about 8% by weight, based on the total weight of the cosmetic composition. A film former is to be understood as meaning those polymers which leave a continuous film on the skin, the hair or the nails during drying. Such film formers can be used in a wide variety of cosmetic agents, such as face masks, make-up, hair fixatives, hair sprays, hair gels, hair waxes, hair treatments, shampoos or nail varnishes. Preferred are those polymers which have sufficient solubility in water, alcohol or water/alcohol mixtures. In this way, appropriate solutions can be produced which can be applied or processed in a simple manner.

In the context of the present disclosure, a film former is understood in particular as meaning those polymers which, when used in from about 0.05 to about 20% by weight (based on the total weight of the cosmetic agent) of aqueous, alcoholic or aqueous-alcoholic solution, are able to be deposited on the hair in the form of a transparent polymer film. The film former is a major component for effecting the shaping. However, the film former is necessarily different from the wax. Examples of a film former are cationic, anionic, nonionic or amphoteric polymers, in particular nonionic, that is, nonionic, polymers.

The film-forming polymer is preferably selected from vinylpyrrolidone-containing polymers. The film-forming polymer is particularly preferably selected from the group includes polyvinylpyrrolidone, vinylpyrrolidone-vinyl acetate copolymer, vinylcaprolactam/VP/dimethylaminoethyl methacrylate copolymer (INCI), VP/DMAPA acrylate copolymer (INCI), triacontanyl PVP (INCI) and mixtures thereof.

Vinylpyrrolidone/vinyl acetate copolymers are preferably used, more preferably vinylpyrrolidone/vinyl acetate copolymers in which the comonomer ratio vinylpyrrolidone (VP) to vinyl acetate (VA) is between about 50 to about 70 and from about 75 to about 25, most preferably from about 60 to about 40. These are sold, for example, under the trade name Luviskol (BASF), for example, Luviskol VA 64 and Luviskol VA 73.

As a further component, the cosmetic composition necessarily contains a wax in a total amount of from about 3 to about 15% by weight, preferably from about 5 to about 12% by weight, more preferably from about 7 to about 10% by weight, based on the total weight of the cosmetic composition. In the context of the present disclosure, a wax is to be understood as meaning a substance which is solid but kneadable at about 20° C., that is, irreversibly plastically deformable, and melts at above about 37° C. without decomposition and can be polished as a liquid. The wax is necessarily different from the film former and the wax is a chemical compound includes more than just carbon and hydrogen atoms or, if the wax is a multicomponent composition, comprises at least one chemical compound includes more than just carbon and hydrogen atoms.

According to a preferred embodiment of the present disclosure, the wax is a first wax comprising an ester of linear or branched saturated or unsaturated C22 to C40 fatty alcohols with a linear or branched saturated or unsaturated C12 to C20 fatty acid, preferably an ester of a linear saturated C28 to C32 fatty alcohol with a linear saturated C14 to C18 fatty acid, and a second wax comprising a linear or branched saturated or unsaturated C22 to C34 fatty acid, preferably a linear saturated C22 to C34 fatty acid; or the wax comprises a first wax having the INCI name synthetic Beeswax and a second wax having the INCI name Copernicia Cerifera Wax. In these alternative embodiments of the present disclosure, the weight ratio of the first wax to the second wax is preferably between about 6 and about 1 and between 1 and about 2, more preferably between about 4 and about 1 and between about 1 and about 1, most preferably between about 2 and about 1.

The wax is preferably carnauba wax, esparto grass wax, Japan wax, cork wax, sugarcane wax, ouricury wax, montan wax, sunflower wax, fruit wax or an animal wax, such as beeswax and another insect wax, spermaceti, shellac wax, wool wax or raffia fat. It can be advantageous to use hydrogenated or hardened waxes. Furthermore, it is also possible to use chemically modified waxes, in particular the hard waxes, for example montan ester waxes, Sasol waxes and hydrogenated jojoba waxes.

Further suitable are the triglycerides of saturated and possibly hydroxylated C16-30 fatty acids, such as hardened triglyceride fats (hydrogenated palm oil, hydrogenated coconut oil, hydrogenated castor oil), glyceryl tribehenate or glyceryl tri-12-hydroxystearate, furthermore synthetic full esters of fatty acids and glycols (for example Syncrowachs™) or polyols having 2-6 C atoms, fatty acid monoalkanolamides having a C12-22 acyl radical and a C2-4 alkanol radical, esters of aromatic carboxylic acids, dicarboxylic acids or hydroxycarboxylic acids (for example, 12-hydroxystearic acid) and saturated and/or unsaturated, branched and/or unbranched alcohols having a chain length of 1 to 80 C atoms, lactides of long-chain hydroxycarboxylic acids and full esters of fatty alcohols and di- and tricarboxylic acids, for example, dicetylsuccinate or dicetyl-/stearyl adipate, and mixtures of these substances.

The wax is particularly preferably made of vegetable waxes, in particular carnauba wax (INCI: Copernicia Cerifera Cera), beeswax (INCI: Beeswax Cera Alba), Petrolatum (INCI) and microcrystalline wax, and in particular mixtures thereof.

As a further component, the cosmetic composition necessarily contains a cosmetic oil in a total amount of from about 0.5 to about 12% by weight, preferably from about 2 to about 10% by weight, more preferably from about 5 to about 8% by weight, based on the total weight of the cosmetic composition. In any case, the term cosmetic oil includes substances which have a solubility of less than about 1 g in about 100 ml of water and are either hydrocarbon-based or silicone-based.

According to a preferred embodiment, the cosmetic oil is a monoester of carboxylic acids comprising 8 C atoms and less with C8 to C16 fatty alcohols, preferably a monoester of benzoic acid and a C8 to C16, more preferably C12 to C15 fatty alcohol, or the cosmetic oil is a synthetic cosmetic oil, preferably a cyclic or linear polyorganosiloxane, more preferably a cyclic or linear polydimethylsiloxane.

Preferred esters include cetyl 2-ethylhexanoate, 2-hexyldecyl stearate (for example, Eutanol™ G 16 S), 2-hexyldecyl laurate, isodecyl neopentanoate, isononyl isononanoate, 2-ethylhexyl palmitate (for example, Cegesoft™ C 24) and 2-ethylhexyl stearate (for example, Cetiol™ 868). Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, di isotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate. Cetyl 2-ethylhexanoate is particularly preferred.

Particularly preferred are benzoic acid C12-C15 alkyl esters, for example, available as a commercial product Finsolv™ TN, benzoic acid isostearyl ester, for example, available as a commercial product Finsolv™ SB, ethylhexyl benzoate, for example, available as a commercial product Finsolv™ EB, and benzoic acid octyldodecyl ester, for example, available as a commercial product Finsolv™ BOD.

If the cosmetic oil is a polyorganosiloxane, a linear or cyclic polydimethylsiloxane is preferred, wherein the linear polydimethylsiloxane more preferably satisfies the formula (CH₃)₃SiO[SiO(CH₃)₂]ₙSi(CH₃)₃ with n=4 to 100, more preferably n=5 to 50, most preferably n=10 to 25, or wherein the cyclic polydimethylsiloxane more preferably has the basic structure [—Si—(CH₃)₂O—]ₙ with n=4 to 12, more preferably n=4 to 8, most preferably n=5. Cyclotetrasiloxane, cyclopentasiloxane, cyclohexasiloxane, cycloheptasiloxane or cyclooctasiloxane, in particular cyclopentasiloxane, are therefore preferably used.

According to a preferred embodiment, the viscosity of the polyorganosiloxane in the cosmetic preparation is from about 1 to about 100 cSt, preferably from about 2 to about 75 cSt, more preferably from about 5 to about 50 cSt. A falling ball viscometer is used to determine the viscosity of the polyorganosiloxane. With its help, its viscosity can be determined using the DIN 53015. To simplify the determination of which polyorganosiloxanes can be used as contemplated herein in the cosmetic preparation, the manufacturer's instructions are used for the viscosity of the polyorganosiloxanes. The polyorganosiloxanes used are all commercially available and usually classified with respect to their viscosities as measured in Stokes.

Further cosmetic oils of natural origin can be used with preference. Preferred can be the use of natural oils, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderflower seed oil, currant seed oil, jojoba oil, linseed oil, macadamia nut oil, corn oil, almond oil, Manila oil, evening primrose oil, olive oil, palm oil, palm kernel oil, Brazil nut oil, pecan oil, peach kernel rapeseed oil, castor oil, sea buckthorn fruit oil, sea buckthorn seed oil, sesame oil, soybean oil, sunflower oil, grapeseed oil, walnut oil, wild rose oil, wheat germ oil, and the liquid portions of coconut oil and the like. However, synthetic triglyceride oils are also preferred, in particular capric/caprylic triglycerides, for example, the commercial products Myritol™ 318, Myritol™ 331 (BASF) or Miglyol™ 812 (Hüls) with unbranched fatty acid radicals and glyceryl triisostearin with branched fatty acid radicals.

It can be extremely preferable to use mixtures of the aforementioned oils.

As a further component, the cosmetic composition necessarily contains a branched or unbranched hydrocarbon in a total amount of from about 5 to about 12% by weight, preferably from about 6 to about 10% by weight, more preferably from about 7 to about 9% by weight, based on the total weight of the cosmetic composition. In the context of the present disclosure, a branched or unbranched hydrocarbon should be understood as meaning substances which have exclusively carbon and hydrogen atoms, or it should be understood as meaning mixtures of substances in which the predominant fraction (greater than about 50% by weight) of the substances includes exclusively carbon and hydrogen atoms.

According to a preferred embodiment of the present disclosure, the branched or unbranched hydrocarbon is a mixture comprising from about 70 to about 90% by weight of a mixture of paraffins and olefins, wherein the mixture has a melting point of from about 38 to about 58° C. Alternatively, the branched or unbranched hydrocarbon is a hydrocarbon mixture having the INCI name Petrolatum.

An essential element of the present disclosure is the choice of waxes and hydrocarbons, in particular their proportion of the total weight, in combination with the film former, which especially contributes to solving the problem underlying the present disclosure. A cosmetic composition having this combination of features is a useful “2 in 1” composition.

As a further component, the cosmetic composition necessarily contains an emulsifier in a total amount of from about 1 to about 4% by weight, preferably from about 2 to about 3% by weight, more preferably from about 1.5 to about 2.5% by weight, based on the total weight of the cosmetic composition.

Preferred emulsifiers are nonionic emulsifiers. A preferred emulsifier is a nonionic surfactant of linear fatty alcohol having ethylene oxide and/or propylene oxide groups. In particular, the emulsifier is a polyalkylene glycol ether of a C12 to C20 fatty alcohol having from 0 to 10, preferably from 0 to 6, propylene oxide groups and from 5 to 30, preferably from 10 to 25, more preferably from 18 to 22 ethylene oxide groups.

Very particular preference is given to mixtures of two emulsifiers. According to a preferred embodiment of the present disclosure, the emulsifier is a mixture of a first polyalkylene glycol ether of a fatty alcohol and a second polyalkylene glycol ether of a fatty alcohol, wherein the first polyalkylene glycol ether of a fatty alcohol is a polypropylene/ethylene glycol ether of a C12 to C20 fatty alcohol having from about 3 to about 7 propylene oxide groups and from 18 to 22 ethylene oxide groups, and the second polyalkylene glycol ether of a fatty alcohol is a polyethylene glycol ether of a C12 to C20 fatty alcohol having from 18 to 22 ethylene oxide groups, wherein preferably the first polyalkylene glycol ether of a fatty alcohol has fewer C atoms in the fatty alcohol component than the second polyalkylene glycol ether of a fatty alcohol.

Examples of particularly preferred nonionic surfactants are compounds having the INCI names Steareth-2, Steareth-21, Oleth-10, PEG-100 Stearate or PPG-5-Ceteth-20 and in particular combinations thereof.

Addition products of ethylene oxide to linear fatty alcohols, to fatty acids, to fatty acid alkloamides, to fatty acid monoglycerides, to sorbitan fatty acid monoesters, to fatty acid glycerides, to methylglucoside monofatty acid esters, to polydimethylsiloxanes and mixtures thereof are useful as nonionic emulsifiers.

Emulsifiers also preferred are the esters of fatty acids having 12 to 18 carbon atoms with saccharides. In particular, the mono- and/or diesters of saccharose (sucrose) with stearic and/or palmitic acid are preferably used. Examples of particularly preferred nonionic emulsifiers are compounds having the INCI names Sucrose Stearate, Sucrose Distearate and mixtures thereof.

Emulsifiers also preferred are addition products of from about 2 to about 20 moles of ethylene oxide to beeswax, in particular compounds having the INCI names PEG-6 Beeswax, PEG-8 Beeswax, PEG-12 Beeswax or PEG-20 Beeswax. PEG-8 Beeswax is particularly preferred from this class of emulsifiers.

A further class of emulsifiers which can be used are the monoesters of fatty acids having 12 to 22 carbon atoms with glycerin. In particular, the monoesters of glycerin with stearic and/or palmitic acid are preferably used. Examples of particularly preferred emulsifiers are compounds having the INCI names Glyceryl Stearate, Glyceryl Palmitate or mixtures thereof.

The preferred emulsifiers stabilize the selected components of the cosmetic compositions to obtain a sufficiently durable agent.

The composition as contemplated herein is necessarily an aqueous composition. It comprises water in a total amount of from about 30 to about 50% by weight, preferably from about 35 to about 48% by weight, more preferably from about 38 to about 41% by weight, based on the total weight of the cosmetic composition.

According to a preferred embodiment of the present disclosure, the cosmetic composition further contains a hydrophobically-modified (meth)acrylic acid copolymer, preferably a copolymer of (meth)acrylic acid and a C10 to C30 alkyl(meth)acrylate, wherein the hydrophobically modified (meth)acrylic acid copolymer is more preferable cross-linked. In the compositions of the present disclosure, these copolymers have proven to be a particularly powerful thickener which are compatible with the other components. In particular, the aqueous composition as contemplated herein obtains an advantageous consistency when using the preferred thickener.

Very particularly preferred cosmetic preparations as contemplated herein comprise at least one of the following embodiments A) to F):

A) Cosmetic composition for the temporary reshaping of keratinic fibers, in particular human hair, containing, based on its total weight, a vinylpyrrolidone/vinyl acetate copolymer as film former in a total amount of from about 1 to about 10% by weight, preferably from about 4 to about 9% by weight, more preferably from about 6 to about 8% by weight, at least one wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight.
B) Cosmetic composition for the temporary reshaping of keratinic fibers, in particular human hair, containing, based on its total weight, a film former in a total amount of from about 1 to about 10%, preferably from about 4 to about 9% by weight, more preferably from about 6 to about 8% by weight, a wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight, wherein the wax is a first wax comprising an ester of linear or branched saturated or unsaturated C22 to C40 fatty alcohols with a linear or branched saturated or unsaturated C12 to C20 fatty acid, preferably an ester of a linear saturated C28 to C32 fatty alcohol with a linear saturated C14 to C18 fatty acid, and a second wax comprising a linear or branched saturated or unsaturated C22 to C34 fatty acid, preferably a linear saturated C22 to C34 fatty acid.
C) Cosmetic composition for the temporary reshaping of keratinic fibers, in particular human hair, containing, based on its total weight, a film former in a total amount of from about 1 to about 10% by weight, preferably from about 4 to about 9% by weight, more preferably from about 6 to about 8% by weight, at least one wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight, wherein the branched or unbranched hydrocarbon is a mixture comprising from about 70 to about 90% by weight of a mixture of paraffins and olefins, wherein the mixture has a melting point of from about 38 to about 58° C.
D) Cosmetic composition for the temporary reshaping of keratinic fibers, in particular human hair, containing, based on its total weight, a film former in a total amount of from about 1 to about 10% by weight, preferably from about 4 to about 9% by weight, more preferably from about 6 to about 8% by weight, a wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight, wherein the cosmetic oil is a cyclic or linear polydimethylsiloxane.
E) Cosmetic composition for the temporary reshaping of keratinic fibers, in particular human hair, containing, based on its total weight, a vinylpyrrolidone/vinyl acetate copolymer as film former in which the comonomer ratio of vinylpyrrolidone to vinyl acetate is at from about 60 to about 40, in a total amount of from about 1 to about 10% by weight, preferably from about 4 to about 9% by weight, more preferably from about 6 to about 8% by weight, a wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight, wherein the wax comprises a first wax having the INCI name synthetic Beeswax and a second wax having the INCI name Copernicia Cerifera Wax, wherein preferably the weight ratio of the first wax to the second wax is between about 6 to about 1 and from about 1 to about 2, and wherein the cosmetic oil is a cyclic or linear polydimethylsiloxane.
F) Cosmetic composition for the temporary reshaping of keratinic fibers, in particular human hair, containing, based on its total weight, a vinylpyrrolidone/vinyl acetate copolymer as a film former in a total amount of from about 1 to about 10% by weight, preferably from about 4 to about 9% by weight, more preferably from about 6 to about 8% by weight, at least one wax in a total amount of from about 3 to about 15% by weight, preferably from about 5 to about 12% by weight, more preferably from about 7 to about 10% by weight, at least one cosmetic oil in a total amount of from about 0.5 to about 12% by weight, preferably from about 2 to about 10% by weight, more preferably from about 5 to about 8% by weight, at least one branched or unbranched hydrocarbon in a total amount of from about 5 to about 12% by weight, preferably from about 6 to about 10% by weight, more preferably from about 7 to about 9% by weight, at least one emulsifier in a total amount of from about 1 to about 4% by weight, preferably from about 2 to about 3% by weight, more preferably from about 1.5 to about 2.5% by weight, and water in a total amount of from about 30 to about 50% by weight, preferably from about 35 to about 48% by weight, more preferably from about 38 to about 41% by weight, wherein the cosmetic composition further contains a hydrophobically modified (meth)acrylic acid copolymer, wherein the hydrophobically modified (meth)acrylic acid copolymer is crosslinked.

The problem underlying the present disclosure is solved by the subject matter of claim 9. A second subject of the present disclosure is therefore the use of the cosmetic composition as contemplated herein for the temporary reshaping and/or shape fixing of keratinic fibers, in particular human hair, preferably for temporary reshaping and/or shape fixing of rigid hair, more preferably for temporary reshaping and/or shape fixing of beard hair.

It has been proven that the composition according to the present disclosure is a useful “2 in 1” composition that is equally suitable for reshaping/shape fixing of human scalp hair and for reshaping/shape fixing of beard hair.

The problem underlying the present disclosure is solved by the subject matter of claim 10. A third subject of the present disclosure is therefore a method for the temporary reshaping of keratinic fibers, in particular human hair, in which the cosmetic composition as contemplated herein is applied to the keratinic fibers, preferably to beard hair, and the keratinic fibers, preferably the beard hair, are subsequently reshaped or fixed.

According to a preferred embodiment, the composition as contemplated herein is applied to the keratinic fibers, that is, to the beard hair and the scalp hair, and the keratinic fibers, that is, the beard hair and the scalp hair, are subsequently reshaped or fixed.

Features relating to preferred embodiments of the first subject of the present disclosure, which are described above only in this regard, of course, apply mutatis mutandis to the second and third subjects as features of preferred embodiments.

The following example is intended to illustrate the subject matter of the present disclosure without limiting it in any way.

### Example

The following preparation as contemplated herein can be prepared:

| Proportion | |
|---|---|
| (in % by weight) | Component (partly as INCI name) |
| 41.0 | Water |
| 0.1 | Acrylates/C10-30 Alkyl Acrylate Crosspolymer |
| 3.0 | Glycerin |
| 1.0 | Panthenol, Aqua |
| 12.0 | Petrolatum |
| 8.0 | Synthetic beeswax |
| 4.0 | Copernicia Cerifera (Carnauba) Wax (Cera Carnauba) |
| 8.0 | C12-15 alkyl benzoates |
| 2.0 | Dimethicone |
| 5.0 | Palmitic Acid, Stearic Acid |
| 1.0 | PPG-5-ceteth-20 |
| 2.0 | Steareth-21 |
| 1.0 | Stearyl Alcohol |
| 2.0 | Perfume (fragrance) |
| 2.0 | Phenoxyethanol, methylparaben, ethylparaben, |
| | propylene glycol |
| 0.4 | Aminomethyl propanol |
| 7.5 | VP/VA Copolymer, Aqua (Water) |
| Σ 100.0 | |

The styling agent is exemplified by a creamy consistency. It can be well introduced into scalp hair and beard hair. The hair can then be shaped well.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic composition for the temporary reshaping of keratinic fibers, comprising based on the total weight of the cosmetic composition,
at least one film former in a total amount of from about 1 to about 10% by weight,
at least one wax in a total amount of from about 3 to about 15% by weight,
at least one cosmetic oil in a total amount of from about 0.5 to about 12% by weight,
at least one branched or unbranched hydrocarbon in a total amount of from about 5 to about 12% by weight,
at least one emulsifier in a total amount of from about 1 to about 4% by weight, and
water in a total amount of from about 30 to about 50% by weight.

2. The cosmetic composition according to claim 1, wherein the film former is a vinylpyrrolidone/vinyl acetate copolymer.

3. The cosmetic composition according to claim 1, wherein the wax is a first wax comprising an ester of linear or branched saturated or unsaturated C22 to C40 fatty alcohols with a linear or branched saturated or unsaturated C12 to C20 fatty acid and a second wax comprising a linear or branched C22 to C34 saturated or unsaturated fatty acid or the wax comprises a first wax having the INCI name synthetic Beeswax and a second wax having the INCI name Copernicia Cerifera Wax, wherein the weight ratio of the first wax to the second wax is between about 6 to about 1 and about 1 to about 2.

4. The cosmetic composition according to claim 1, wherein the branched or unbranched hydrocarbon is a mixture comprising about 70 to about 90% by weight of a mixture of paraffins and olefins and the mixture has a melting point of about 38 to about 58° C. or the branched or unbranched hydrocarbon is a hydrocarbon mixture having the INCI name Petrolatum.

5. The cosmetic composition according to claim 1, wherein the cosmetic oil is a monoester of carboxylic acids comprising 8 C atoms and less with C8 to C16 fatty alcohols or the cosmetic oil is a synthetic cosmetic oil.

6. The cosmetic composition according to claim 1, wherein the emulsifier is a nonionic surfactant of a linear fatty alcohol having ethylene oxide and/or propylene oxide groups or the emulsifier is a polyalkylene glycol ether of a C12 to C20 fatty alcohol having 0 to 10 propylene oxide groups and 5 to 30 ethylene oxide groups.

7. The cosmetic composition according to claim 1, wherein the emulsifier is a mixture of a first polyalkylene glycol ether of a fatty alcohol and a second polyalkylene glycol ether of a fatty alcohol, wherein the first polyalkylene glycol ether of a fatty alcohol is a polypropylene/ethylene glycol ether of a C12 to C20 fatty alcohol having 3 to 7 propylene oxide groups and 18 to 22 ethylene oxide groups, and the second polyalkylene glycol ether of a fatty alcohol is a polyethylene glycol ether of a C12 to C20 fatty alcohol having 18 to 22 ethylene oxide groups, and wherein the first polyalkylene glycol ether of a fatty alcohol has fewer C atoms in the fatty alcohol component than the second polyalkylene glycol ether of a fatty alcohol.

8. The cosmetic composition according to claim 1, further comprising a hydrophobically modified (meth)acrylic acid copolymer.

9. A use of the cosmetic composition according to claim 1 for temporary reshaping and/or shape fixing of keratinic fibers.

10. A method for the temporary reshaping of keratinic fibers comprising:
applying the cosmetic composition according to claim 1 to the keratinic fibers; and
subsequently reshaping or fixing the keratinic fibers.

11. The cosmetic composition according to claim 2 wherein the vinylpyrrolidone/vinyl acetate copolymer has a comonomer ratio of vinylpyrrolidone to vinylacetate of between about 50 to about 70.

12. The cosmetic composition according to claim 3 wherein the weight ratio of the first wax to the second wax is about 2 to about 1.

13. The cosmetic composition according to claim 5 wherein the cosmetic oil is a monoester of benzoic acid and a C12 to C15 fatty alcohol.

14. The cosmetic composition according to claim 5 wherein the cosmetic oil is a cyclic or linear polydimethylsiloxane.

15. The cosmetic composition according to claim 8 wherein the hydrophobically modified (meth)acrylic acid copolymer is crosslinked.

16. The cosmetic composition according to claim 8 wherein the hydrophobically modified (meth)acrylic acid copolymer is further defined as a copolymer of (meth)acrylic acid and a C10 to C30 alkyl(meth)acrylate.

17. The cosmetic composition according to claim 1 wherein:
the at least one film former is present in a total amount of from about 6 to about 8% by weight based on the total weight of the cosmetic composition;
the at least one wax is present in a total amount of from about 7 to about 10% by weight based on the total weight of the cosmetic composition;
the at least one cosmetic oil is present in a total amount of from about 5 to about 8% by weight based on the total weight of the cosmetic composition;
the at least one branched or unbranched hydrocarbon is present in a total amount of from about 7 to about 9% by weight based on the total weight of the cosmetic composition;
the at least one emulsifier is present in a total amount of from about 1.5 to about 2.5% by weight based on the total weight of the cosmetic composition; and
the water is present in a total amount of from about 38 to about 41% by weight based on the total weight of the cosmetic composition.

18. A cosmetic composition for the temporary reshaping of keratinic fibers comprising, based on the total weight of the cosmetic composition,
a vinylpyrrolidone/vinyl acetate copolymer present in a total amount of from about 1 to about 10% by weight;
a first wax having the INCI name synthetic Beeswax and a second wax having the INCI name Copernicia Cerifera Wax, wherein the first and second waxes together is present in a total amount of from about 3 to about 15% by weight;
a cosmetic oil chosen from a C12 to C15 alkyl benzoate, a polydimethylsiloxane, and combinations thereof, wherein the cosmetic oil is present in a total amount of from about 0.5 to about 12% by weight;
Petrolatum present in a total amount of from about 5 to about 12% by weight;
at least one emulsifier present in a total amount of from about 1 to about 4% by weight; and
water present in a total amount of from about 30 to about 50% by weight.
